(19) United States
(12) Patent Application Publication
(10) Pub. No.: US 2008/0215106 A1
(43) Pub. Date: Sep. 4, 2008

(54) Thoracoscopically implantable diaphragm stimulator

(76) Inventors: Chang Lee, Redwood City, CA (US); Amir J. Tehrani, San Francisco, CA (US); David Ligon, San Francisco, CA (US); Rose Province, San Jose, CA (US)

Correspondence Address: RMX, L.L.C., P.O. Box 3550, Los Altos, CA, 94024, US

(21) Appl. No.: 12/069,823

(22) Filed: Feb. 13, 2008

Related U.S. Application Data
(63) Continuation-in-part of application No. 12/004,932, filed on Dec. 21, 2007.
Application No. 12/004,932 is a continuation-in-part of application No. 11/981,342, filed on Oct. 31, 2007.
Application No. 11/981,342 is a continuation-in-part of application No. 11/480,074, filed on Jun. 29, 2006.
Application No. 11/480,074 is a continuation-in-part of application No. 11/271,726, filed on Nov. 10, 2005.
Application No. 11/271,726 is a continuation-in-part of application No. 10/966,484, filed on Oct. 15, 2004.
Application No. 10/966,484 is a continuation-in-part of application No. 10/966,474, filed on Oct. 15, 2004.
Application No. 10/966,474 is a continuation-in-part of application No. 10/966,421, filed on Oct. 15, 2004.
Application No. 10/966,421 is a continuation-in-part of application No. 10/966,472, filed on Oct. 15, 2004.
Application No. 10/966,472 is a continuation-in-part of application No. 10/686,891, filed on Oct. 15, 2003.
(60) Provisional application No. 60/901,154, filed on Feb. 13, 2007.

(51) Int. Cl. A61N 1/365; A61N 1/36; A61N 1/05; A61B 5/0488

(52) U.S. Cl. 607/17; 607/42; 607/116; 600/546

(57) ABSTRACT

A diaphragm stimulator is provided to treat one or more diseases disorders or conditions where the stimulator is configured to be implanted by way of a thoracoscopic approach to the diaphragm. The stimulator may include sensors positioned within the thorax. The stimulator may also include or be used with a cardiac rhythm management device.

### RELATED APPLICATION DATA

This application claims priority of Provisional Application No. 60/901,154 and is a continuation in part of U.S. application Ser. No. 12/004,932 filed Dec. 21, 2007, which is a continuation in part of U.S. application Ser. No. 11/981,342 filed Oct. 31, 2007 which is a continuation in part of U.S. application Ser. No. 11/480,074 filed Jun. 29, 2006 which is a continuation in part of U.S. application Ser. No. 11/271,726 filed Nov. 10, 2005 which is a continuation in part of U.S. application Ser. No. 10/966,484 filed Oct. 15, 2004; U.S. application Ser. No. 10/966,474, filed Oct. 15, 2004; U.S. application Ser. No. 10/966,421, filed Oct. 15, 2004; and U.S. application Ser. No. 10/966,472 filed Oct. 15, 2004 which are continuations in part of U.S. application Ser. No. 10/686,891 filed Oct. 15, 2003 entitled: BREATHING DISORDER DETECTION AND THERAPY DELIVERY DEVICE AND METHOD all of which are incorporated in their entirety without limitation, herein by reference.

### FIELD OF THE INVENTION

This application relates to a diaphragm stimulator for use in stimulating the diaphragm and/or phrenic nerve to provide a therapy to treat one or more diseases disorders or conditions.

### BACKGROUND OF THE INVENTION

Diaphragm and/or phrenic nerve stimulation have been proposed for a number of therapeutic applications. Diaphragm stimulators have been implanted as phrenic nerve stimulators in the neck region and in the thorax region using nerve cuffs about the left or right main branch of the phrenic nerve. Diaphragm stimulators have also been implanted directly on the abdominal side of the diaphragm. These stimulators use laparoscopic techniques to place the diaphragm stimulator through the abdomen.

Abdominal implanted diaphragm stimulators may require an additional step of mapping to identify the phrenic nerve motor points or to otherwise identify an ideal placement for the stimulator.

It would be desirable to provide an alternative diaphragm stimulation device. It would also be desirable to provide a diaphragm stimulation device and implantation method that would not require increased surgical time for mapping to identify desired electrode implant sites.

### SUMMARY OF THE INVENTION

In accordance with one aspect of the invention treatment may be provided for number of diseases, disorders and conditions that may relate to, have co-morbidities with, affect, be affected by respiratory or lung health status, respiration, ventilation, or blood gas levels. Such diseases and disorders may include but are not limited to obstructive respiratory disorders, upper airway resistance syndrome, snoring, obstructive apnea; central respiratory disorders, central apnea; hypopnea, hypoventilation, obesity hypoventilation syndrome other respiratory insufficiencies, inadequate ventilation or gas exchange, chronic obstructive pulmonary diseases; asthma; emphysema; chronic bronchitis; circulatory disorders; hemodynamic disorders; hypertension; heart disease; chronic heart failure; cardiac rhythm disorders; obesity or injuries in particular affecting breathing or ventilation.

A number of these treatments and diaphragm stimulators are described in the related applications set forth in the related application data above, all of which are incorporated herein by reference.

In accordance with the invention a thoracoscopically implanted device is provided. In a procedure in accordance with the invention, two or more sets of access ports or holes may be formed the chest region for instrument access through the diaphragm and electrode placement on the thoracic side of the diaphragm.

In accordance with the invention endoscopes may be used to visualize and identify phrenic nerve structures and branches for electrode placement. In accordance with the invention a phrenic nerve motor point may be visually identified.

An electrode or electrodes configured to be implanted at the diaphragm may be provided in accordance with the invention.

An electrode or electrodes may be configured to be implanted on or adjacent one or more nerve branches entering into the diaphragm

An electrode or electrodes may be configured to be implanted on the diaphragm adjacent a plurality of nerve branches entering into the diaphragm.

A plurality of electrodes may be configured to be implanted on or adjacent a plurality of phrenic nerve sub-branches to permit selective, sequential or titrated activation of one or more portions of the diaphragm.

The electrode assembly may include a pressure sensor to measure thoracic pressure to obtain flow information based on thoracic pressure. A movement or contraction sensor may be provided with the electrode assembly as well. The movement or contraction sensor may be used to sense diaphragm movement, diaphragm contraction and/or other patient movement or movement artifacts. A diaphragm movement/contraction sensor may be configured to be positioned on the diaphragm.

An electrode (either the stimulation electrode or a separate electrode) may also be used to sense EMG. Accordingly, the sensors and stimulating electrodes may be provided on a single assembly. Alternatively, separate sensors may be used as well. The sensors may be used to obtain various diagnostic information. Accordingly, a single procedure may be used to implant thoracic pressure sensors and diaphragm EMG sensors, as well as electrode(s) that may be used for stimulation and/or sensing. Further a single device may contain all of these components. The sensor or sensors may be positioned on the diaphragm or in the thoracic cavity. A sensor or stimulator may also be placed in or on the chest wall from within the thorax to sense one or more parameters (e.g., EMG, chest wall movement, or pressure) or to stimulate chest wall movement. Stimulation may be provided to elicit movement of the external intercostals, the levator costae muscle and/or the parasternal intercostals.

Sensing the diaphragm EMG may be used to provide information on the electrical activity of the diaphragm and electrical characteristics of inspiration cycle on breath by breath basis. Diaphragm EMG may also be used to sense movement by sensing lower frequencies. Conducting EMG frequency analysis may lead to identification and delivery of a more optimum therapy, for example, by comparing EMG frequency content to a baseline to determine one or more conditions or respiration parameters, e.g., among other things, respiratory effort or frequency changes indicative of a breathing disorder or a precursor to a disordered event. Examples of uses and methods relating to detection and analysis of EMG are set forth in related applications hereto which have been incorporated in their entirety without limitation herein.

Sensing diaphragm movement may provide close monitoring of the mechanical characteristics of the inspiration and exhalation cycles. Diaphragm movement sensing may provide direct information on rate and magnitude of exhalation. Intrathoracic pressure is correlated with lung volume and accordingly may be used to determine functional residual capacity changes. Accordingly, the invention provides a device that may be implanted using a single surgical access procedure, that may be used to obtain information from diaphragm activity in conjunction with thoracic pressure. Such information may be used, for example, in detection strategies/techniques relating to breathing abnormalities or disorders. Furthermore, such device provides improved sensing/monitoring of the responses/results of the stimulation utilizing another sensor such as diaphragm movement and or thoracic pressure sensor. Sensors placed on the chest wall may also be used to detect chest wall movement or activation. Lung volume and paradoxical rib cage movement among other things, may be detected. These parameters may be used to determine information including, but not limited to, detecting breathing disorders and determining therapeutic efficacy. Such sensing and feedback may be used in diaphragm or phrenic nerve stimulation devices without limitation to location of stimulation electrodes, external or implanted in one or more locations of the body.

In accordance with another aspect of the invention diaphragm stimulation is provided in combination with a cardiac rhythm management device. A thoracoscopic approach to implanting a diaphragm stimulation device also provides access to the heart for lead implantation. For example, it has been proposed to put leads on the left side of the heart for biventricular pacing. Also, epicardial leads may be placed around the heart for defibrillation. Also sensing, pacing, and defibrillation may be performed from a location outside of the heart and through a thoracoscopic approach.

### BRIEF DESCRIPTION OF THE DRAWINGS

FIG. 1A is a schematic illustration of a patient undergoing a thoracoscopic procedure to implant a thoracoscopic diaphragm stimulator.

FIG. 1B is a schematic illustration of a thoracoscopically implanted diaphragm stimulator and a cardiac rhythm management device.

FIGS. 2A to 2E illustrate an electrode being attached to a diaphragm from a thoracoscopic approach.

FIG. 3 is a schematic illustration of a thoracoscopically implanted device in accordance with the invention.

FIG. 4 is a schematic illustration of a thoracoscopically implanted device in accordance with the invention.

FIG. 5 is a schematic illustration of a thoracoscopically implanted device in accordance with the invention.

### DETAILED DESCRIPTION OF THE DRAWINGS

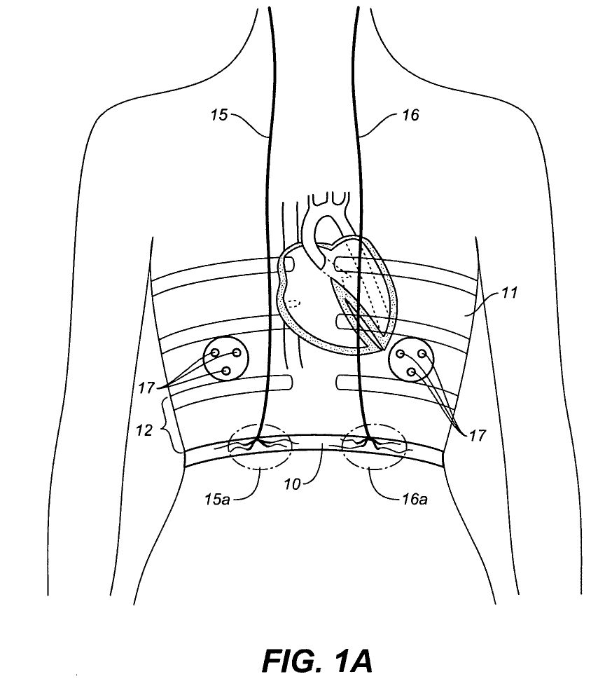

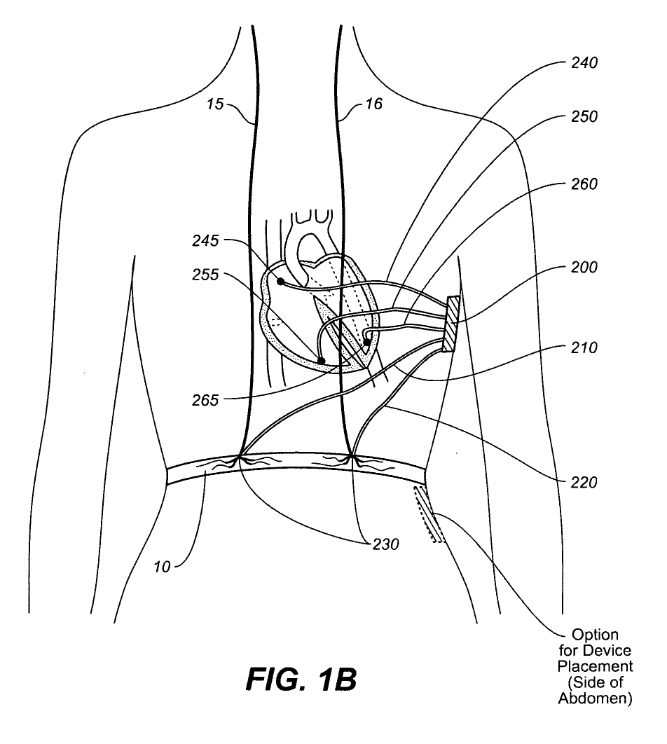

Referring to FIG. 1A, a right branch 15 and left branch 16 of a phrenic nerve are illustrated. At the lower portion 12 of the thoracic region 11, the phrenic nerve branches 15, 16 branch off to sub-branches 15a and 16a into the tissue associated with the diaphragm 10.

As shown in FIG. 1A, port holes 17 or formed through the chest between ribs using thoracoscopic surgical techniques. Port holes 17 are illustrated on each side of the patient to accordingly access each on the phrenic nerve branches 15, 16 (one on each hemidiaphragm). Alternatively ports 17 may used on one side of the patient to access each hemidiaphragm where the mediastinum is taken down or cut through to access the hemidiaphragm on the opposite side.

According to one aspect insufflation is used to provide visualization and access to the diaphragm 10 and the lower phrenic nerve branches at the lower portion of the diaphragm 10. According to another aspect, the lung may be collapsed to provide visualization and access to the diaphragm.

During the procedure each lung may be selectively ventilated while placing the electrode on the opposite side.

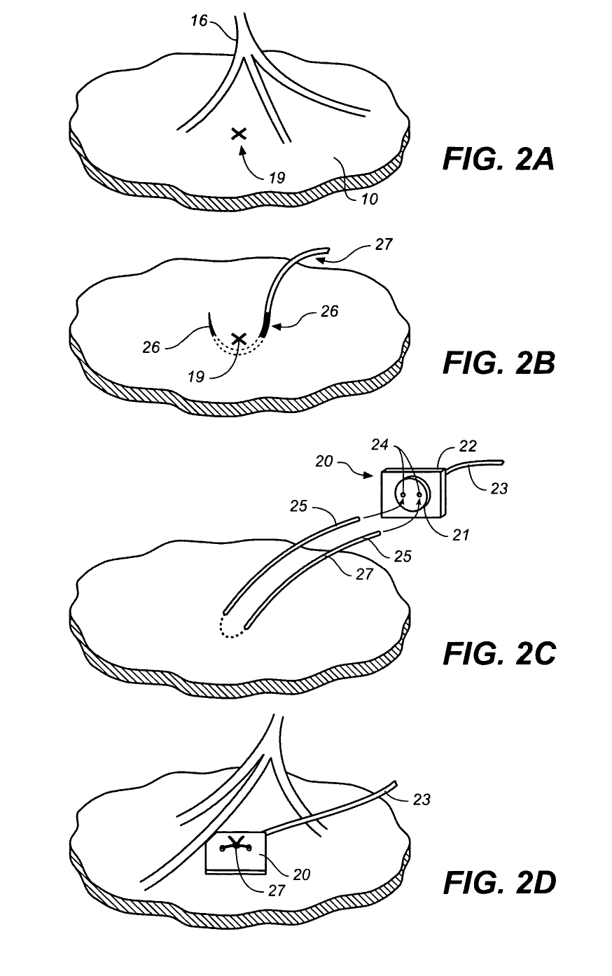

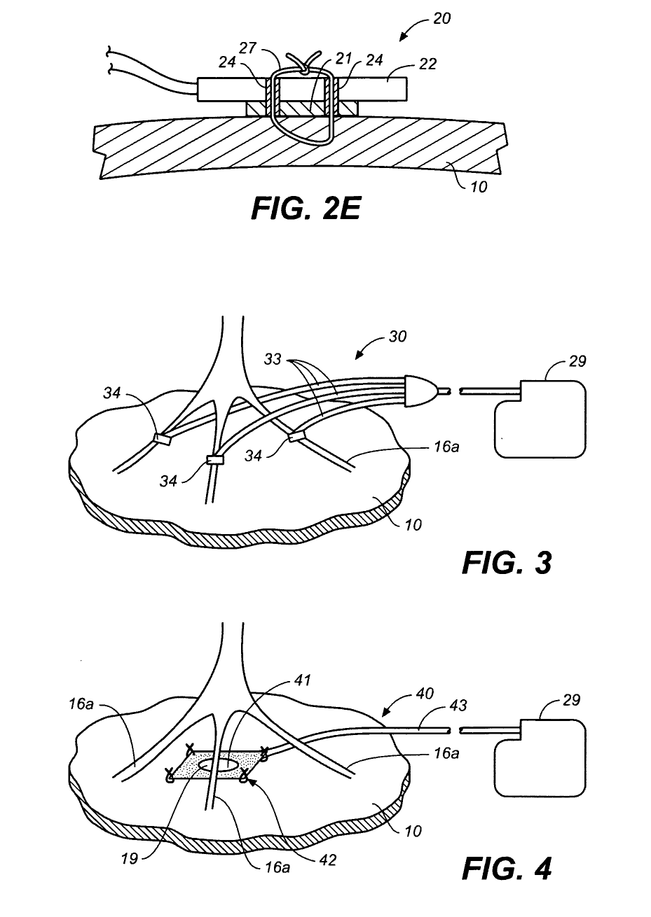

Referring to FIGS. 2A to 2E a procedure is illustrated whereby an electrode is attached to a diaphragm using a thoracoscopic approach. As illustrated in FIG. 2A the phrenic nerve 16 splits into sub-branches 16a as it enters into the diaphragm 10. A location at or adjacent a motor point 19 for electrode placement may be identified visually as a location on the diaphragm between one or more branches entering into the diaphragm.

As illustrated in FIG. 2B, a suture 27 is provided at the motor point 19 using a suture needle 26. Alternative devices for positioning a suture or tether at the motor point 19 may be used as well.

As illustrated in FIG. 2C an electrode assembly 20 comprises and electrode 21, an electrode support structure 22 on which electrode 21 is positioned, a lead 23 extending from the assembly 20, and openings 24 for receiving the suture 27. The two ends 25 of the placed suture 27 are inserted into the openings 24 of the electrode assembly 20.

As illustrated in FIG. 2D, the electrode assembly 20 is guided with the suture 27 to the motor point 19 where the electrode assembly 20 is secured with the suture to the diaphragm 10. The electrode 21 and support structure 22 are configured, i.e., sized and shaped so that they may be positioned on the diaphragm on the motor point 19 in an area defined by the entrance of the sub branches 16a into the diaphragm 10.

FIG. 2E is a schematic cross section illustrating the electrode assembly 20 of FIGS. 2A-2D attached to the diaphragm with the anchoring suture 27.

The electronics unit or pulse generator may be subcutaneously placed and attached to lead 23 extending from electrode 21 and support structure 22.

Referring to FIG. 3, an electrode assembly 30 of a stimulation device is illustrated implanted in the thorax. The electrode assembly 30 comprises a plurality of nerve cuffs 34 coupled to leads 33 which are coupled to a pulse generator 29. The nerve cuffs 34 are attached to sub-branches 16a of the phrenic nerve branch 16.

Referring to FIG. 4, an electrode assembly 40 comprising an electrode 41 and support structure 42, is illustrated sutured on its outer periphery to the diaphragm 10. The electrode 41 is positioned on the motor point 19 of the diaphragm within an area defined by the nerve sub-branches 16a of the phrenic nerve entering into the diaphragm 10. A lead 43 extends out of the electrode assembly to a pulse generator.

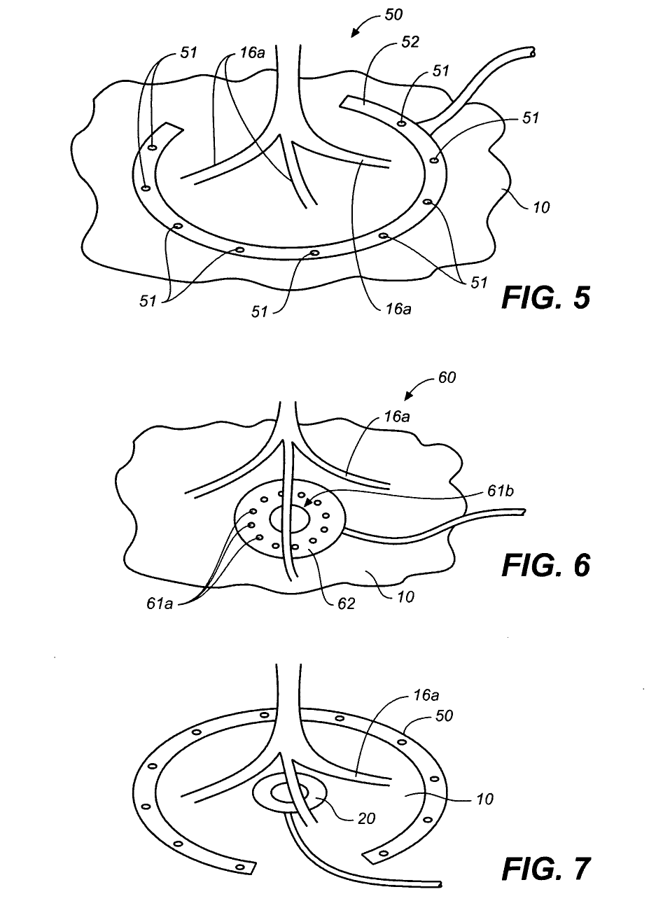

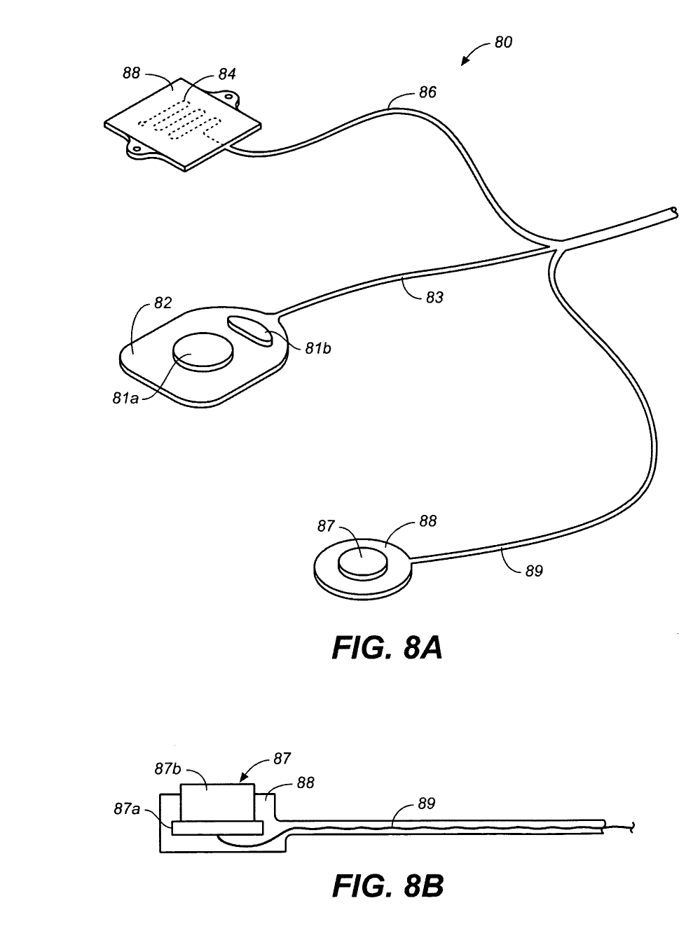

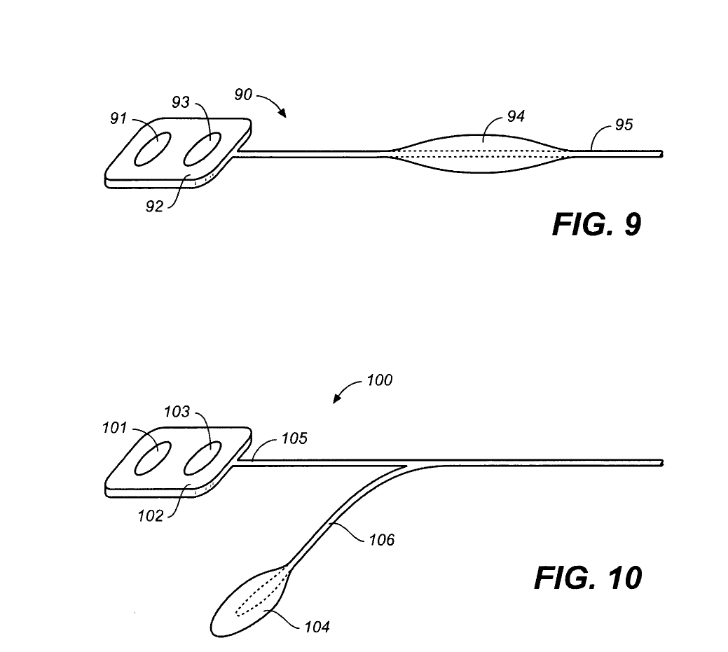

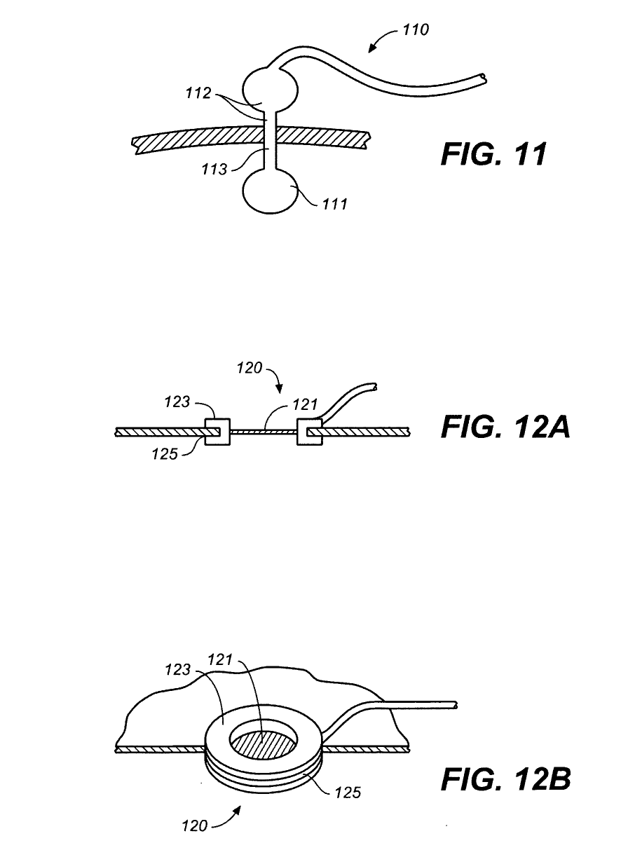

Referring to FIG. 5, an electrode assembly 50 is illustrated comprising a plurality of electrodes 51 positioned around an electrode support structure 52. The support structure is configured to be positioned on the diaphragm 10 and about the periphery of at least a portion of the nerve sub-branch structures 16a entering into the diaphragm 10. Accordingly, one or more electrodes may be activated or selected to stimulate one or more nerve branches. As such stimulation may be provided to individual nerve sub-branches or to a plurality of nerve sub-branches by selecting electrodes and/or by providing stimulation in a sequence. Accordingly stimulation may be provided to activate the diaphragm in order to increase functional residual capacity. Stimulation may be provided to gradually activate nerves, e.g. in a sequence. Stimulation may be provided where the entire diaphragm is not activated simultaneously. Also, stimulation may be altered from one electrode to another to provide rest to one or more nerve sub-branches and/or one or more portions of the diaphragm controlled by one or more nerve sub-branches, and thus reduce fatigue and/or adaptation to stimulation. The electrode support structure 52 is illustrated in a broken peripheral form thereby permitting placement of the support structure around a plurality of the nerve sub branches 16a as they enter into the diaphragm 10. Accordingly, the stimulation may activate one hemidiaphragm at the time for fatigue resistant or therapeutic purposes.

FIG. 6 illustrates an electrode assembly 60 comprising a plurality electrodes 61a located peripherally on a support structure 62 and a motor point stimulation electrode 61b located in the middle of the support structure 62. The support structure 62 is sized and configured to fit in an area defined by the nerve sub-branches 16a entering into the diaphragm 10. The motor point stimulation electrode 61b and peripheral electrodes 61a may be selected based on a stimulation protocol and/or based on feedback indicating diaphragm response to stimulation. For example, the motor point stimulation electrode 61b may be used for stimulation to activate generally the entire diaphragm, e.g., FRC increase, for diaphragm pacing and/or breathing control. The peripheral electrodes 61a may be used to stimulate individual branches or groups of branches of the nerves in a manner similar to that described with respect to electrodes 51 as set forth with respect to FIG. 5. In addition, the peripheral electrodes may be used to activate the generally the entire diaphragm. A number of protocols may be used such as, for example, stimulation techniques and protocols described in related applications set forth above and incorporated herein by reference. Various electrodes may be selected or used in accordance with such protocols.

FIG. 7 illustrates a combined use of electrode assembly 20 illustrated in FIG. 2E and electrode assembly 50 illustrated in FIG. 5. Similar to electrode assembly 60 described with respect to FIG. 6, a combination of electrode assemblies 20 and 50 may act as motor point electrodes and peripheral electrodes respectively and may be selected in a manner similar to motor point electrode 61b and peripheral electrodes 61a as described with respect to FIG. 6.

FIG. 8A illustrates an electrode/sensor assembly 80 comprising: a stimulation and/or sensing electrodes 81a and 81b positioned with a support structure 82 and having a lead 83 extending therefrom; a strain gauge or a contraction or movement sensor 84 positioned with a support structure 85 and having a lead 86 extending therefrom; and a pressure sensor 87 positioned with a support structure 88 and having a lead 89 extending therefrom. The leads 83, 86 and 89 may be connected to a subcutaneously implanted pulse generator. The support structure 82 and stimulation electrode 81 may be positioned on the diaphragm 10 for stimulation. The strain gauge 84 and support structure 85 may be positioned on the diaphragm to provide information concerning diaphragm movement or information concerning contraction and relaxation of the diaphragm, e.g., from which inspiratory effort may be determined as well as parameters associated with exhalation including exhalation rate. The strain gauge or other movement or contraction sensor on the diaphragm may also be used to determine effect of the stimulation, for example, the magnitude of a breath or functional residual capacity (FRC) change. Alternatively or additionally, the strain gauge and support structure may be positioned, on the chest wall to obtain information concerning chest wall movement. An ultrasonic micrometer may be used on the diaphragm or chest wall to determine movement of the diaphragm or chest wall. The micrometer may be a passive device that may be interrogated. If used in conjunction with diaphragm EMG, paradoxical motion during breathing or stimulation may be sensed. The paradoxical motion may indicate obstruction or other breathing inefficiency. Electrodes may be placed on the inner chest wall. Impedance plethysmography may be used sensing impedance changes at the chest wall. The impedance changes may be used among other things to determine lung volume. The pressure sensor 87 may be positioned or attached within the intrapleural space to provide information concerning intrapleural pressure. Intrapleural pressure may be correlated with lung volume as well as level of inspiratory effort (i.e., diaphragm and chest wall muscle activity)

FIG. 8B is a schematic side cross-sectional view of the pressure sensor 87. The pressure sensor 87 includes a transducer 87a for pleural pressure sensing within a silicone support structure 88 and having a porous steroid eluting plug 87b positioned over the pressure sensor 87. The steroid helps prevent encapsulation over the pressure sensor 87.

FIG. 9 illustrates an electrode/sensor assembly 90 comprising an electrode support structure 92 including an electrode 91 that may be used for stimulation or EMG sensing and a diaphragm movement sensor 93. The electrode/sensor assembly 90 further comprises a balloon catheter 94 for sensing pleural pressure. A lead 95 extends from the support structure 92. The balloon catheter 94 is also positioned on the lead 95. In use, the support structure 92 is attached to the diaphragm 10 while the balloon catheter 94 remains freely within the intrapleural space. Alternatively, the balloon catheter may be sutured to the diaphragm or chest wall within the intrapleural space.

FIG. 10 illustrates and electrode/sensor assembly 100 comprising and electrode support structure 102 including an electrode 101 that may be used for stimulation or EMG sensing and a diaphragm movement sensor 103. The electrode/sensor assembly 100 further comprises a balloon catheter 104 for sensing pleural pressure. A lead 105 extends from the support structure 102. The balloon catheter 10 is positioned on lead 106 extending therefrom so that the pressure sensor 104 may be position remotely from the assembly 102 which is attached to the diaphragm.

FIG. 11 illustrates a differential pressure sensor assembly 110 implanted in a diaphragm and comprising an abdominal pressure sensor 111 located on a first side of the sensor assembly 110 and a pleural pressure sensor 112 located on a second side of the sensor assembly. An elongate portion extends between the first side and the second side whereby the elongate portion 113 extends through the diaphragm. The differential pressure measurement provides more reliable data concerning flow and diaphragm movement. Other body movement causing noise in the signal may generally be cancelled out by using the difference in pressure between the diaphragm abdominal side and the diaphragm pleural side. The pressure sensor may be delivered into position across the wall of the diaphragm, e.g. using a needle or cannula. The differential pressure across the diaphragm is a measure of true force exerted by the diaphragm. This may be used, e.g., to determine effort, changes in functional residual capacity, and/or to measure diaphragm fatigue.

FIGS. 12A and 12B illustrate an alternative differential pressure sensor assembly 120. The assembly 120 comprises a ring portion 123 with a notch 125 formed about the ring for receiving and coupling to a diaphragm. A diaphragm type differential pressure sensor 121 is positioned inside the ring for sensing the difference in pressure between the abdominal side of the diaphragm and the pleural side of the diaphragm.

FIG. 1B illustrates a combined diaphragm stimulator and CRM device in accordance with the invention which may include, for example, a bradypacemaker, ICD (implantable cardioverter defibrillator), CRT-D (cardiac resynchronization therapy defibrillator) or a CRT-P (pacemaker). A pulse generator 200 is implanted subcutaneously, for example, in a region under the patient's arm. Leads 210, 220 from the pulse generator are coupled to electrode assemblies 230, on right and left hemidiaphragms. The electrode assemblies 230 are implanted thoracoscopically on or at diaphragm 10, for example, as described herein with respect to one or more of FIGS. 1A, or FIGS. 2A to 12B. Leads 240, 250, and 260 from the pulse generator 200 are respectively coupled to right atrial electrode 245, right ventricular and defibrillator electrode 255, and left ventricular electrode 265 for CRT (cardiac resynchronization therapy) pacing. The pulse generator 200 may combine diaphragm pacing and cardiac rhythm management. The advantages of combining the CRM with a thoracoscopically implanted diaphragm stimulator are that one device may monitor and deliver therapy for treatment of cardiac arrhythmias and breathing disorders among patients with heart failure or other cardiovascular diseases. Breathing therapy alone (for example as described in one or more related applications as set forth herein) may also improve the health of cardiovascular and hypertension patients. Many of the cardiovascular patients have some sort of breathing disorder. In accordance with the invention, a combined CRM device and breathing therapy device may be used to treat cardiovascular conditions. Furthermore, breathing disorder detection may be used to predict cardiac arrythmias whereby preemptive therapy may be provided either by stimulating the diaphragm or by stimulating the heart.

A single pulse generator unit may be used for both applications whereby a lead to the heart may be coupled to the pulse generator at a time after the diaphragm stimulation lead has been in place and in use, or visa versa.

Separate pulse generators may also be used. For example, if a patient has already received a CRM device, can also receive a breathing therapy device through the thoracoscopic, abdominal, or pectoral procedures and the two devices may be synchronized utilizing ECG as the global signal. Such therapies may include for example breathing therapies described in the related applications as set forth herein, all of which are incorporated in their entirety, without limitation, herein. In accordance with this aspect of the invention ECG may be sensed using one or more electrodes placed on the diaphragm, chest wall or heart. Also the diaphragm stimulation device may be programmed to detect arrhythmias or other cardiac status so that cardiac and respiratory therapies would not interact negatively. For example, for a particular vulnerable cardiac status, respiratory therapy may be disabled. Or if appropriate for a particular cardiac status, respiratory therapy may be enabled. Thus the breathing disorder device may be programmed to insure that there are no negative device/device interactions. One example may be the sensing of a diaphragm stimulation artifact and preventing the artifact from being mistaken for cardiac arrhythmia. Separate cardiac and respiratory devices may communicate with each other, for example through RF or Bluetooth.

1. A method for providing diaphragm stimulation comprising:
accessing the thoracic side of a diaphragm of a subject;
positioning an electrode on the diaphragm;
providing stimulation from a signal source through electrode to electrically stimulate the diaphragm.

2. The method of claim 1 wherein the step of positioning the electrode on the diaphragm comprises positioning the electrode near a phrenic nerve branch and diaphragm junction.

3. The method of claim 1 further comprising
sensing intrathoracic pressure from within the thoracic cavity and setting a stimulation parameter based on intrathoracic pressure.

4. The method of claim 1 further comprising:
sensing diaphragm movement with a movement sensor positioned on the diaphragm from within the thoracic cavity.

5. The method of claim 1 further comprising sensing intercostal movement.

6. The method of claim 1 wherein the step of positioning the electrode adjacent a phrenic nerve motor point comprises positioning a plurality of electrodes adjacent a plurality of phrenic nerve branches.

7. A diaphragm stimulation device comprising:
an electrical stimulator comprising:
an electrode configured to be positioned on a thoracic side of a diaphragm;
a signal source configured to supply a stimulating signal through the electrode.

8. The diaphragm stimulation device of claim 7 further comprising:
an intrathoracic pressure sensor coupled to the stimulator.

9. The diaphragm stimulation device of claim 7 further comprising and intrathoracic pressure sensor.

10. The diaphragm stimulation device of claim 7 wherein the device is further configured to be coupled to a cardiac rhythm management device.

11. The diaphragm stimulation device of claim 7 further comprising a movement sensor.

12. A device for detecting paradoxical motion comprising:
a first sensor configured to sense diaphragm movement, wherein the first sensor is configured to be implanted on a diaphragm of a subject;
an second sensor configured to sense chest wall movement from within a chest wall of a subject; and
a processor configured to receive a first signal from the first sensor and a second signal from the second sensor; wherein the processor is configured to determine presence of paradoxical motion from the first and second signals.

13. A device for providing cardiac rhythm management and diaphragm stimulation comprising:
a cardiac rhythm management device configured to be implanted within a thorax of a subject; and
a diaphragm stimulation device configured to be implanted within a thorax of a patient;
wherein the cardiac rhythm management device and the diaphragm stimulation device are configured to be coupled together within the thorax.

14. The device of claim 13 wherein the cardiac rhythm management device and the diaphragm stimulation device are combined into a device.

15. An electrode for positioning on a diaphragm of a subject comprising:
a first electrode portion configured to be positioned on a diaphragm about at least a portion of a phrenic nerve branch of a subject.

16. The electrode of claim 15 further comprising a second electrode portion configured to be positioned within an area circumscribed by entry of phrenic nerve branches entering into a diaphragm.

17. An electrode for positioning on a diaphragm of a subject comprising:
a first electrode portion configured to be positioned within an area circumscribed by entry of phrenic nerve branches entering into a diaphragm.

18. A method for implanting an electrode on a diaphragm comprising:
thoracoscopically accessing a diaphragm of a subject;
positioning an electrode on the diaphragm from within the thorax of the subject.

19. The method of claim 18 wherein the step of positioning the electrode comprises positioning the electrode on the diaphragm about at least a portion of a phrenic nerve branch of a subject.

20. The method of claim 18 wherein the step of positioning the electrode comprises positioning the electrode on the diaphragm within an area circumscribed by entry of phrenic nerve branches entering into a diaphragm.

21. A device for detecting breathing disorders or determining therapeutic efficacy comprising:
an EMG sensor configured to sense an EMG signal of a diaphragm;
a signal processor configured to process an EMG signal to determine frequency components of the EMG signal; and
a processor configured to determine a characteristic of breathing from the frequency components.

22. The device of claim 21 wherein the processor is configured to determine therapeutic efficacy of stimulation from the frequency components.

23. The device of claim 21 wherein the processor is configured to detect a breathing disorder based on the frequency components.
